(19) United States
(12) Patent Application Publication
(10) Pub. No.: US 2026/0335497 A1
(43) Pub. Date: Oct. 29, 2026

(54) HYDROGEL EMBOLICS AND NON-THERMAL TREATMENTS USING THE SAME

(71) Applicant: Bard Peripheral Vascular, Inc., Franklin Lakes, NJ (US)

(72) Inventors: Song Ding, Apex, NC (US); Cameron James Bloomquist, Pittsboro, NC (US)

(21) Appl. No.: 19/186,976

(22) Filed: Apr. 23, 2025

(51) Int. Cl. A61L 24/00; A61L 24/04; A61L 24/08; A61L 24/10

(52) U.S. Cl. CPC A61L 24/0031; A61L 24/043; A61L 24/08; A61L 24/102; A61L 24/104; A61L 24/108

(57) ABSTRACT

Embodiments relate to hydrogel embolic compositions having modifiers containing catechol moieties and a one-pot synthesis for forming the same. The hydrogel embolic can include a complexing components bound to adhesive components, where the adhesive components carry the modifier having the at least one catechol moiety.

### BACKGROUND

Chronic venous insufficiency (CVI) is a leading vascular disease with clinical manifestations including varicose veins, edemas, venous ulcers, and venous hypertension. A non-thermal therapeutic approach to CVI involves using a catheter to introduce cyanoacrylate-based medical adhesive, causing diseased veins to collapse and form scar tissue. Adverse events to cyanoacrylate-based adhesives can include hypersensitivity reactions, foreign body reactions, and skin perforation and extrusion of the permanent, rigid material. These adverse events can require a surgical vein excision if severe.

However, such techniques are often plagued by issues of over or under delivery of agents to the target tissue, such as a tumor.

### SUMMARY

Embodiments of the present disclosure relate to a one-pot synthesis of injectable modified hydrogel embolics for tissue treatments, the hydrogel embolic, kits for making and using the same, and methods for making and using the same.

In an embodiment, a hydrogel embolic is disclosed. The hydrogel embolic includes an adhesive component bonded to at least one complexing component. The adhesive component includes a core including a glycerol base, at least one linker bound to the core where the at least one linker includes a polyethylene glycol (PEG), and at least one modifier bonded to the at least one linker, the at least one modifier including at least one catechol moiety. The at least one complexing component includes a polyamine.

In an embodiment, the core includes one or more of pentaerythritol, hexaglycerol, tripentaerythritol, or glycerol. In an embodiment, the at least one modifier includes dopamine or dopamine hydrochloride. In an embodiment, the polyamine includes one or more of human serum albumin, recombinant human serum albumin, bovine serum albumin, poly lysine, or spermidine. In an embodiment, the adhesive component includes a multi-arm PEG-n-hydroxy succinimide (NHS) with dopamine bound thereto.

In an embodiment, a kit for dispensing a hydrogel embolic into a human in disclosed. The kit includes a first group of components including a polyamine, a base, and a first aqueous solution having a pH of about 8.0 or more. The kit includes second group of components including a multi a multi-arm PEG having succinimidyl ester tethering groups, at least one modifier including a catechol moiety, and a second aqueous solution.

In an embodiment, the first group of components may include albumin as the polyamine, sodium carbonate as the base, and water to form a first solution including the same with a pH between 8.0 and 12.0. In an embodiment, the second group of components may include a multi-arm PEG n-hydroxy succinimide (NHS) ester, dopamine hydrochloride, and water to form a first solution having the same in an acidic solution with a pH between 2.0 and 6.0.

In an embodiment, a method of treating a veinous condition in a human body is disclosed. The method includes forming a hydrogel embolic including an adhesive component and at least one complexing component bound to the adhesive component. The adhesive component includes a core having a glycerol base; at least one linker bound to the core, the at least one linker including a polyethylene glycol (PEG); and at least one modifier bonded to the at least one linker, the at least one modifier including at least one catechol moiety. The at least one complexing component includes a polyamine. The method includes delivering the hydrogel embolic at least locally within the human body via an embolization. The method includes adhering the hydrogel embolic onto a vein surface within the human body.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a schematic of a one-pot synthesis of a modified PEG hydrogel embolic, according to an embodiment.

FIG. 2 is top view of a kit for forming and delivering a hydrogel embolic, according to an embodiment.

FIG. 3 is a flow chart of a method of treating a veinous condition in a human body, according to an embodiment.

### DETAILED DESCRIPTION

Embodiments of the present disclosure relate to a one-pot synthesis of injectable modified hydrogel embolics for tissue treatments, the hydrogel embolic, kits for making and using the same, and methods for making and using the same. The modified hydrogel embolic includes an adhesive component and at least one complexing component. The adhesive component includes a core, at least one linker bound to the core, the at least one linker including a polyethylene glycol (PEG), and at least one modifier bonded to the at least one linker, the at least one modifier including at least one catechol moiety. The at least one complexing component includes a polyamine and is bound to the adhesive component to form the hydrogel embolic. K its for forming the hydrogel embolic include the components of the adhesive component and the complexing component, and may further include containers, mixing means, and delivery means (e.g., dual syringe apparatus). Methods for making the hydrogel embolic include forming the hydrogel embolic (e.g., mixing the components) contemporaneously with delivery of the hydrogel embolic into the human body

The injectable modified hydrogel embolics include a biocompatible moiety for bonding to tissues in the body. For example, a hydrogel embolic having components with catechol moieties may adhere to tissues in the body (e.g., blood vessel surface to occlude vessel for CVI treatment) and provide a biodegradable material that avoids the pitfalls of conventional materials used to treat CVI.

Arterial embolization is designed to fill target vessels to occlude flow, thereby limiting nutrient and oxygen supply to the target area (e.g., varicose vein). However, conventional therapeutic approaches to CVI involve using a catheter to introduce cyanoacrylate-based medical adhesive, causing diseased veins to collapse and form scar tissue. Adverse events to cyanoacrylate-based adhesives can include hypersensitivity reactions, foreign body reactions, and skin perforation and extrusion of the permanent, rigid material. These adverse events can require a surgical vein excision if severe.

The modified hydrogels disclosed herein include a PEG-based hydrogel embolic having a modifier bound thereto, where the modifier includes at least one catechol moiety.

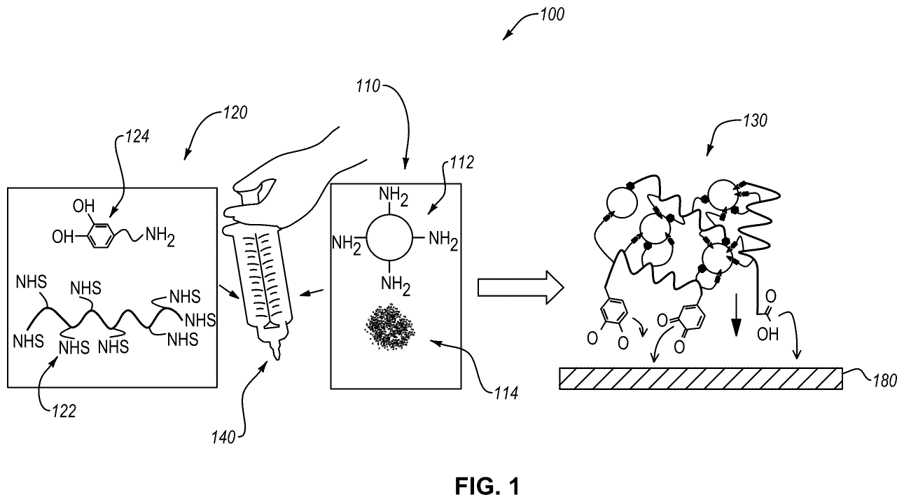

FIG. 1 is a schematic of a one-pot synthesis method 100 for forming a modified PEG hydrogel embolic, according to an embodiment. As shown in FIG. 1, under the method 100 a first group of components 110 is mixed with a second group of components 120 to form a hydrogel embolic 130. As will be discussed in more detail below, the first group of components 110 and the second group of components 120 are composed to form the hydrogel embolic 130 after mixing the groups of components in a one “pot” (e.g., single step) synthesis.

The hydrogel embolic 130 (e.g., PEG-based dopamine-modified hydrogel embolic) includes an adhesive component (122 bonded to 124) and a complexing component 112 which may be combined in a one-pot synthesis to form the hydrogel embolic 130. The hydrogel embolic 130 may be used more broadly as a hydrogel adhesive. In some examples, the hydrogel embolic 130 may include a PEG-based hydrogel embolic having one or more modifiers with catechol moieties bound thereto. The one or more modifiers may include dopamine. Components used to form the hydrogel embolic 130 are disclosed in more detail below.

In some embodiments, the hydrogel embolic (e.g., embolic) may include a hydrogel having at least one complexing component and at least one adhesive component bonded to the at least one complexing component to form a hydrogel backbone. The adhesive component of the hydrogel embolic 130 may be formed by bonding a multi-arm PEG having tethering groups 122 with at least one modifier 124 having at least one catechol moiety. The at least one complexing component 112 may include a polyamine (e.g., an albumin). The hydrogel embolic 130 is formed by bonding the adhesive component (including at least one modifier having at least one catechol moiety) to the at least one complexing component. The at least one modifier may include dopamine.

The complexing component 112 may include one or more polyamines. For example, the one or more polyamines may include one or more of human serum albumin, recombinant human serum albumin, bovine serum albumin, polylysine, or spermidine. The complexing component 112 may include one or more peptides, polypeptides, or proteins. A peptide includes to two or more amino acids linked via a peptide bond, a polypeptide includes a series or chain of multiple linked amino acids, and a protein refers to a full-length expressed gene or chimera thereof. In some examples, the complexing component 112 may include a protein, such as a recombinant protein and/or synthetic protein. The protein may include a commonly found protein in humans. Such common protein(s) may be desirable because they produce limited response by the immune system of humans. In some examples, the complexing component includes an albumin, such as human albumin, human serum albumin (HSA), recombinant human serum albumin (rHSA), synthetic human serum albumin, or the like. In some examples, the complexing component 112 may include poly-lysine or a polypeptide with multiple lysine residues. Such examples include amine groups as reactive side chains available to react with (e.g., cross-link to) the adhesive component (e.g., PEG-NHS ester of the adhesive component).

In some examples, the complexing component 112 (e.g., complexing molecule) has a molecular weight (M W) of about 1,000 Da to about 100,000 Da, such as about 1,000 Da to about 20,000 Da, about 20,000 D a to about 50,000 Da, about 50,000 Da to about 70,000 Da, about 60,000 Da to about 80,000 Da, about 70,000 Da to about 100,000 Da, less than 100,000 Da, or less than 70,000 Da. For example, rHSA has a MW of about 66 kDa. A poly-lysine polypeptide M W can be constructed based on a desired size or M W. Such a M Ws may be similar or identical to the any of the M Ws disclosed above with respect to albumin.

The complexing component 112 may be provided in an amount effective to result in a (hydrated) hydrogel embolic 130 having about 2 to about 60 weight % (wt %) complexing component, such as about 2 wt % to about 10 wt %, about 3 wt % to about 8 wt %, about 5 wt % to about 15 wt %, about 15 wt % to about 25 wt %, about 10 wt % to about 20 wt %, about 15 wt % to about 35 wt %, about 20 wt % to about 40 wt %, about 30 wt % to about 50 wt %, about 40 wt % to about 60 wt %, less than 60 wt %, less than 40 wt %, or less than 10 wt % of the hydrogel embolic.

The complexing component 112 may be provided in dry form and may be hydrated before or contemporaneous with deployment of the hydrogel embolic into the human body. For example, the complexing component may include an rHSA powder. The complexing component may be disposed in a basic solution, such as at a pH of at least 8.0, such as 8.0 to 10.0, 9.0 to 10.0, at least 8.5, at least 9.0, or at least 9.5.

The adhesive component (e.g., a multi-arm PEG having tethering groups 122 bonded to the at least one modifier 124) is formulated or otherwise configured to bond with (e.g., cross-link to) the at least one complexing component 112. Accordingly, when reacted together the at least one adhesive component and the at least one complexing component 112 cross-link to form the hydrogel backbone of the hydrogel embolic.

The adhesive component may include a plurality of chemical species (e.g., monomers, oligomers, or polymers) composed to bond with the complexing component. As explained in more detail below the adhesive component may include a PEG, such as a multi-arm PEG having one or more tethering groups thereon. The one or more tethering groups are composed to bond with (e.g., cross-link with) one or more functional groups of the complexing component.

The adhesive component(s) may include at least one linker (or a spacer) with moieties along the length thereof for binding to other adhesive components, complexing components 112, or water. For example, the branching component(s) may include a PEG or repeating units thereof, attached to a core or central moiety at one end and a tethering group at the opposing end.

Formula I below is an example structure of the a multi-arm PEG having tethering groups 122 of the adhesive component:

### (Core)-(Linker)ₙ-(Tethering Group) (I)

The number n may vary depending on the desired final molecular mass of the branching component or resulting hydrogel. For example, n may be an integer from 1 to 1,000, such as 1 to 100, 100 to 200, 200 to 300, 300 to 400, 400 to 500, 500 to 1,000, at least 20, at least 25, at least 40, at least 50, at least 75, at least 100, less than 1,000, less than 500, less than 200, less than 100, or less than 50.

The core may include a hydroxyl or polyol, such as pentaerythritol, hexaglycerol, tripentaerythritol, or glycerol. In some examples, the core is glycerol-based core allowing for multiple “arms” to extend therefrom. For example, a glycerol may provide for two arms, pentaerythritol can provide up to four arms, hexaglycerol can provide up to eight arms, and tripentaerythritol can provide up to eight arms.

The linker provides a connection between the tethering group and the core. The linker may include an ethylene glycol, a PEG, or a linear or branched chain thereof. For example, a PEG-chain may link the tethering group to the core. The linker may be a PEG sufficient to provide a molecular weight (M W) of at least about 1,000 Daltons (Da) to the PEG or the resulting branching component, such as about 1,000 Da to about 100,000 Da, about 1,000 Da to about 10,000 Da, about 10,000 Da to about 20,000 Da, about 15,000 Da to about 25,000 Da, about 20,000 Da to about 40,000 Da, about 40,000 Da to about 60,000 Da, about 60,000 Da to about 80,000 Da, about 80,000 Da to about 100,000 Da, about 1,000 Da to about 50,000 Da, about 50,000 Da to about 100,000 Da, less than about 100,000 Da, or less than about 50,000 Da. Selective control of the M W of the PEG in the linker can allow for the hydrogel to retain a selected amount of water, such as a greater M W corresponding to a greater amount of water retained.

The linker may have one or more functional groups or moieties at the terminal ends thereof composed to bond with the tethering group and core. The linker may be composed to bond with a reactive moiety of the tethering group to form the branching component. Such reactive moieties may include an amine, an alkene, an alkyne, or the like. In some examples, the linker may include one or more carbonyl containing group(s), such as at least one carboxylic acid, ester, acid halide, aldehyde, or the like.

The tethering group of the multi-arm PEG having tethering groups 122 is composed to covalently bond with one or more moieties of the complexing component and the modifier 124 upon mixing.

The tethering group may include an ester comprising the carbonyl group or ester of the linker. For example, the tethering group may include a succinimidyl ester of the PEG linker. In such examples, the succinimide may be N-hydroxy succinimide (NHS). The succinimide may be a further substituted NHS. The tethering group may include a succinimidyl carboxymethyl (SCM), a succinimidyl succinate (SS), a succinimidyl glutarate (SG), or the like. In some examples, the tethering group includes at least one of NHS-SG, NHS-SS, and NHS-SCM. The tethering group may include a maleimide (e.g., maleimide bonded to the PEG linker), propionamide (e.g., propionamide bonded to the PEG linker).

In some examples, the tethering group may include a cyclical or ringed organic compound conjugated to the linker through a carboxyl group or an appended carboxyl group of a carboxylic acid. For example, a succinimide is bonded to the carboxylic acid via the nitrogen heteroatom in the five-member ring. In some examples, the tethering group may include BA, NHS, acrylamide, an α-haloacetyl, biotin, a carbodiimide, a carboxylic acid, an alkyne, a halogen (such as chloride), an epoxide, hydrazide, norborene, hydroxyl, azide, amine, acrylate, dibenzocyclooctyne (DBCO), glutamic acid, glutaramide acid, a succinimide (e.g., succinimidyl glutarate (SG), succinimidyl glutaramide (GAS)), a maleimide, para-nitrophenyl carbonate (NPC), a propionamide, a pyridyl disulfide (e.g., orthopyridyl disulfide (OPSS)), acetic acid, carboxyl methyl, glutaric acid, succinic acid, glutaramide acid, succinamide acid, succinimidyl succinamide (SAS), succinimidyl carboxymethyl (SCM) ester, thiol (or SH), tosylate, vinylsulfone, or combinations thereof. The tethering group may include esters or any of the foregoing.

In some examples, the tethering group is linked to an alkanedioic acid, such as a linear dicarboxylic acid. In some examples, the linear dicarboxylic acid is saturated. In some examples, the dicarboxylic acid is unsaturated. The alkanedioic/alkenedioic/branched acid may include one or more of oxalic acid, malonic acid, succinic acid, itaconic acid, glutaric acid, adipic acid, pimelic acid, suberic acid, azelaic acid, tartaric acid, malic acid, or citraconic acid. The primary amine or heteroatom of a ringed organic compound (e.g., succinimide) is linked to the dicarboxylic acid. In some examples, the tethering group is an ester of SG or NHS and one of glycolic acid, succinic acid, and glutaric acid.

The linker and/or tethering group may provide reactive moieties for cross-linking with other linkers, tethering groups, or the complexing component, such as exposed oxygens, nitrogens, sulfurs, hydroxyl groups, carbonyl carbons, amines, and the like. For example, the linker may include one or more ethylene glycol molecules or PEGs.

The adhesive component may include a multi-arm molecule, such as a multi-arm PEG. One or more linkers may bind to the multi-arm core and extend outward therefrom. Two, four, six, or eight linkers may be attached to the core. Each linker connected to the core may be considered as an appendage or arm extending therefrom. At least some of the linkers in the multi-arm PEG may include the tethering group bonded thereto.

While described above as having the configuration of a core-linker-tethering group, the multi-arm PEG having tethering groups 122 of the adhesive components may have multi-armed configuration having a core linked to two or more linkers. For example, the branching component may have the core including 2 arms with the structure of:

### (Tethering Group)-(Linker)-(Core)-(Linker)-(Tethering Group) (II).

In such a two-armed structure, a single ethylene glycol molecule may function as the core.

The multi-arm PEG having tethering groups 122 of the adhesive component may have a 4-armed configuration. For example, the core may have 4 arms and have a structure of:

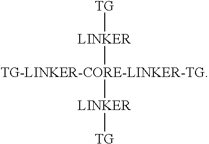

The multi-arm PEG having tethering groups 122 of the adhesive component may have an 8-armed configuration. For example, the core may have 8 arms and have a structure of:

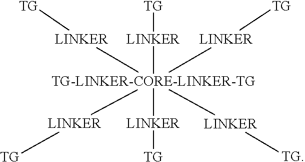

While the cores disclosed herein allow for three, four, five, six, seven, or eight arms (e.g., linkers), the number of arms can be further increased through additional modifications to the core. For example, PEG molecules can be pre-reacted to provide branched structures that each provide an available oxygen for each arm to attach.

In some examples, the adhesive component may include a multi-arm (e.g., 2, 4, or 8-arm) core having PEG linkers, and having tethering groups on one or more of the linkers. The one or more tethering groups may include any of the tethering groups one or more of NHS, NHS-SCM, NHS-SS, or NHS-SG. In specific examples, the branching component includes an 8-arm PEG having tethering groups including one or more of NHS, NHS-SCM, NHS NHS-SS, NHS-SG, or an ester of any of the foregoing including a portion of the linker (e.g., PEG-NHS ester).

The at least one adhesive component may have a molecular weight of at least about 1,000 Da (g/mol), such as about 1,000 Da to about 50,000 g/mol, about 1,000 Da to about 10,000 g/mol, about 10,000 Da to about 30,000 Da, 15,000 Da to about 25,000 Da, about 20,000 Da to about 40,000 Da, about 30,000 Da to about 50,000 Da, less than about 50,000 Da, or less than 30,000 Da.

In some examples, the adhesive component and resulting hydrogel may include a multi-arm PEG having tethering groups 122 with tethering groups of a succinimidyl glutarate, a PEG linker, and a glycerol or glycerol derived core. The succinimidyl glutarate can be an NHS-SG. The multi-arm PEG having tethering groups 122 of the adhesive component can include 2, 4, or 8 arms, such as NHS-PEG-SG-2, NHS-PEG-SG-4, and NHS-PEG-SG-8, respectively.

In some examples, the adhesive component and resulting hydrogel may include the multi-arm PEG having tethering groups 122 with tethering groups of a succinimidyl carboxymethyl, a PEG linker, and a glycerol or glycerol derived core. The succinimidyl carboxymethyl can be an NHS-SCM. The adhesive component can include 2, 4, or 8 arms or NHS-PEG-SCM-2, NHS-PEG-SCM-4, and NHS-PEG-SCM-8, respectively.

In some examples, the adhesive component and resulting hydrogel may include the multi-arm PEG having tethering groups 122 with tethering groups of a succinimidyl succinate (SS), a PEG linker, and a glycerol or glycerol derived core. The succinimidyl succinate can be an NHS-SS. The adhesive component can include 2, 4, or 8 arms or NHS-PEG-SS-2, NHS-PEG-SS-4, and NHS-PEG-SS-8, respectively.

The adhesive component has at least one modifier 124 including at least one catechol moiety. The at least one modifier 124 is carried by the multi-arm PEG having tethering groups 122 such as being bonded, entangled, graphed, encapsulated, or otherwise disposed thereon or therein. The at least one modifier 124 provides a hydrogel embolic that both bonds to wet tissues within the body (e.g., vein, artery), provides a non-thermal treatment for CVI, and is resorbable in the body, such as through hydrolytic degradation.

The at least one modifier 124 may include one or more organic molecules having at least one catechol moiety therein. For example, the at least one modifier 124 may include a catechol amine, such as dopamine, polydopamine, norepinephrine, or the like. The at least one modifier 124 may include N-hydroxyl succinimide esters of any carboxylic acids containing a catechol or gallate moiety, such as NHS ester of caffeic acid, NHS ester of gallic acid, NHS ester of L-DOPA, or the like. The at least one modifier 124 can be tethered to the multi-arm PEG having tethering groups and/or to complexing component(s) by reacting with chemical species therein (e.g., polyamines) to form a bond therebetween.

The modifier 124 may be present in an amount that greater than 0 wt % of the multi-arm PEG having tethering groups used to form the adhesive component, such as about 0.01 wt % to 30 wt %, about 0.1 wt % to about 20 wt %, 1 wt % to 10 wt %, 10 wt % to 20 wt %, about 20 wt % to about 30 wt %, less than 30 wt %, less than 20 wt %, or less than 10 wt % of the multi-arm PEG having tethering groups used to form the adhesive component. More of the modifier 125 may be used to provide more bonding material to bond to tissue surfaces within a human body, such as in inner surface of a vein.

The amount of time required for hydrolytic degradation of the hydrogel embolic can be tuned by using selected cores and linkers in the adhesive component. For example, a two armed core is expected to result in a hydrogel embolic 130 having a two-week period before hydrolytic degradation removes at least 50% of the hydrogel embolic 130. Similarly, an eight armed core is expected to result in a hydrogel embolic 130 having at least a six week period before hydrolytic degradation removes at least 50% of the hydrogel embolic 130. Further tuning of the hydrogel embolic may be accomplished by selectively using longer or shorter PEG chains, more or less cross-linking between PEG chains and the complexing component 112, or both.

The adhesive component is provided in an amount effective to result in a hydrogel embolic 130 with about 2 to about 50% weight/volume (w/v) of the adhesive component, such as about 2% to about 10% w/v, about 3% to about 8% w/v, about 5% to about 15% w/v, about 15% to about 25% w/v, about 10% to about 20% w/v, about 15% to about 35% w/v, about 20% to about 40% w/v, about 30% to about 50% w/v, less than 50% w/v, or less than 30% w/v, or less than 10% w/v.

The adhesive component may be provided in dry form and may be hydrated before or contemporaneous with deployment of the hydrogel embolic (e.g., embolic) into the human body. The one or more sub-components of the adhesive component may be disposed in an aqueous solution, such as water (e.g., an acidic solution, basic solution, both), saline, or the like. The water of the aqueous solution may be of sufficient quality or purity to prevent or limit unwanted contaminants when placed within a tissue 180 of the human body. Such pure water may reduce the risk of generating or exaggerating an immune response in the human body. Such water May include one or more of filtered water, deionized water, distilled water, reverse osmosis water, or the like. Such water may be used in the acid solution used to hydrate the multi-arm PEG having tethering groups 122 and the at least one modifier 124 having one or more catechol moieties, and in the basic solution used to hydrate the complexing component 112.

As shown in FIG. 1, one or more sub-components of the hydrogel embolic 130 may be provided from the first group of components 110 and the second group of components 120.

The first group of components 110 and the second group of components 120 are combined to form the hydrogel embolic 130 (e.g., hydrogel adhesive). For example, the first group of components 110 and the second group of components 120 may be combined to form the hydrogel embolic including the adhesive component and the complexing component 112. The complexing component 112 may include a polyamine (e.g., HSA), where the polyamine is bonded to the adhesive component. The adhesive component may include a core having a glycerol base, at least one linker (e.g., PEG) bound to the core, and at least one modifier bonded to the at least one linker, wherein the at least one modifier (e.g., dopamine) includes at least one catechol moiety.

The first and second groups of components may include one or more sub-components of the hydrogel embolic. For example, the first group of components 110 may include the complexing component (e.g., polyamine), a base (e.g., sodium carbonate), and a first aqueous solution.

The first group of components 110 may include the complexing component 112, such as any of the polyamines disclosed herein in any of the amounts disclosed herein. For example, the polyamine may include one or more of human serum albumin, recombinant human serum albumin, bovine serum albumin, poly-lysine, or spermidine.

The base may be provided in dry form or an aqueous solution. The base may be formulated and present in an amount to selected provide a first aqueous solution having a pH of about 8.0 or more when hydrated by a selected amount of water, such as 8.0 to 12.0, 9.0 to 10.0, 8.0 to 10.0, 9.0 to 11.0, 10.0 to 12.0, less than 11.0, or a pH of at least 9.0. For example, the base may include one or more of sodium carbonate, sodium bicarbonate, calcium carbonate or any other base formulated to provide a basic solution with any of the pHs disclosed above and that is safe to inject into a human subject. The basic solution stabilizes the complexing component 112 (e.g., HSA) and accelerates a reaction between the PEG-NHS esters (of the adhesive component) with the complexing component 112 and the at least one modifier 124 having one or more catechol moieties, when mixed.

The second group of components 120 may include the multi-arm PEG having tethering groups 122, the at least one modifier 124, and a second aqueous solution. The multi-arm PEG having tethering groups 122 may include any of the multi-arm PEG having tethering groups disclosed herein in any of the amounts disclosed herein. For example, the multi-arm PEG having tethering groups 122 may include a multi-arm (PEG) having succinimidyl ester tethering groups such as a 2-arm, a 4-arm, or an 8-arm PEG-NHS ester. The multi-arm PEG having tethering groups 122 may be provided in a powder or a solution.

The at least one modifier 124 includes one or more catechol moieties, such as any of the modifiers disclosed herein in any of the amounts disclosed herein. The at least one modifier may include an acidic component, such as an acidic salt. For example, the at least one modifier may include a hydrochloride or the like. In some examples, the at least one modifier includes dopamine or dopamine hydrochloride. The acidic component may be formulated to provide a pH below 6.0 to the resulting aqueous solution having the at least one modifier 124 and multi-arm PEG having tethering groups bound thereto, such as a pH of 2.0 to 6.0, 2.0 to 4.0, 3.0 to 5.0, 4.0 to 6.0, less than 5.0, or greater than 4.0.

The second solution may include water. The amount of water may be selected to provide a (second) aqueous solution having any of the acidic pHs disclosed above.

In some examples, the first aqueous solution may be composed to have a pH between about 8.0 and 12.0 (e.g., 9.0 to 10.0) after the first group of components are mixed and the second aqueous solution is composed to have a pH between about 2.0 and 6.0 (e.g., 4.0 to 6.0) after the second group of components are mixed.

In some examples, the first aqueous solution (and first group of components) and the second aqueous solution (and second group of components) are composed to form a hydrogel embolic solution with a pH of at least about 8.0 (e.g., 8.0 to 12.0, 8.0 to 10.0, at least 9.0, 9.0 to 11.0, or 9.0 to 10.0) after the first group of components and the second group of components are mixed with each other. For example, upon mixing the first set of components 110 and the second set of components 120, the complexing component 112 and adhesive component (multi-arm PEG having tethering groups 122 bonded to the modifier 124) respectively therein bond to each other (e.g., polymerize, cross-link) to form the hydrogel embolic 130. The tethering group may be bonded or otherwise attached to the at least one modifier 124. In some examples, the tethering group may be at least partially reacted with one or more of the modifier 124 or the complexing component 112, such as entangled with or otherwise physically bound to the modifier or complexing component of the hydrogel embolic.

As shown in more detail below, the first and second group of components 110 and 120 may be provided in separate containers. The separate containers may include the double syringe apparatus 140 shown in FIG. 1. The double syringe apparatus 140 may be used to contemporaneously form and deliver the hydrogel embolic into a selected region in the human body, such as in one or more veins. Sub-components of the hydrogel embolic may be provided in separate containers, such as individually, in one or more groups (e.g., first and second component groups), or both. Such components may be organized in a kit for forming and delivering the hydrogel embolic.

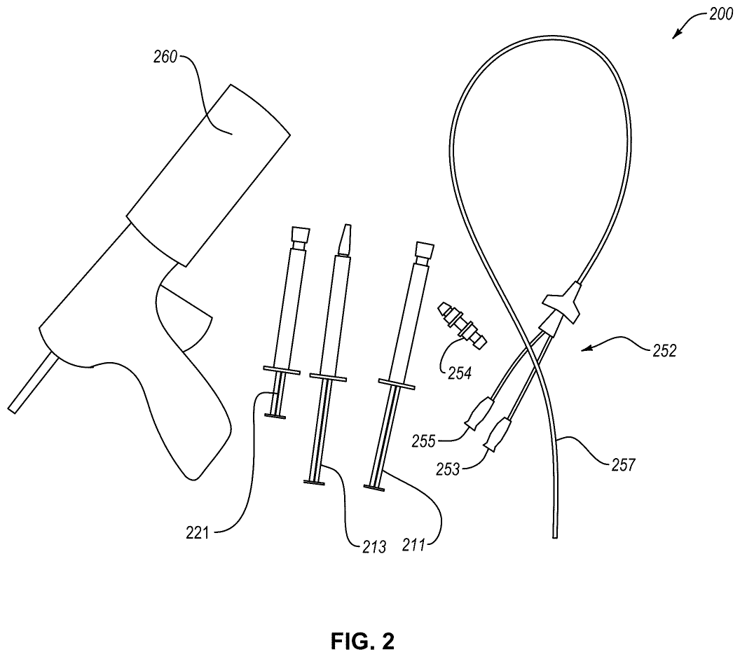

FIG. 2 is top view of a kit 200 for forming and delivering the hydrogel embolic, according to an embodiment. The kit 200 includes a first container 211, a second container 221, a an additional container 213 (e.g., of solvent such as water), a dual lumen connector 252 having connectors 253 and 255 configured to fluidly connecting the first container 211 and the second container 221, a syringe connector 254, and a dual syringe adapter 260 configured to hold and actuate two syringes simultaneously. The kit 200 includes one or more of the components of any of the hydrogel embolics disclosed herein, such as a PEG-NHS ester, a modifier, at least one complexing component, a solvent (e.g., water), a base, an acid, an acidic solution, a basic solution, or combinations of any of the foregoing.

The first container 211 may includes one or more of a vial, a specimen container, a syringe, or the like. The first container 211 may include at least some of the first group of components therein. For example, the first container 211 may include the polyamine, base, water (in basic solution) therein. In some examples, the polyamine and base may be present in the container in dry form, which may be hydrated by a selected amount of water to form the first aqueous solution having the polyamine (e.g., HSA) therein. The first group of components may be provided in the kit 200 in amounts selected to provide the correct amount of components to form a hydrogel embolic having a selected weight or relative amount of modifier (e.g., dopamine) therein.

The first container 211 may include a first syringe as shown. The polyamine and base may be present in the syringe in dry form or in an aqueous solution having a pH above 8.0 as disclosed herein. In some examples, the first container 211 may include a vial, sample container, or the like.

The second container 221 may includes one or more of a vial, a specimen container, a syringe, or the like. The second container 221 may include at least some of the second group of components therein. For example, the second container 221 may include one or more of the PEG-NHS ester, the modifier having the at least one catechol moiety, a dry acid, or water (in acidic solution) therein. In some examples, the PEG-NHS ester, the modifier having the at least one catechol moiety, acid (e.g., dopamine hydrochloride), may be present in the second container in dry form, which may be hydrated by a selected amount of water to form the second aqueous solution (e.g., acidic solution) having the adhesive component therein. The second components may be provided in the kit 200 in amounts selected to provide the correct amount of components to form a hydrogel embolic having a selected weight or relative amount of modifier (e.g., dopamine) therein.

The second container 221 may include a second syringe as shown. The PEG-NHS ester, the modifier having the at least one catechol moiety, and acid may be present in the syringe in dry form or in an aqueous solution having a pH of 6.0 or less as disclosed herein. In some examples, the second container 221 may include a vial, sample container, or the like.

One or more components may be provided in additional containers 213, such as one or more additional syringes. Such additional syringes may include a syringe containing water to form one or more of the first aqueous solution having the polyamine and a pH of 8.0 or more, or the second aqueous solution having the adhesive component (e.g., PEG-NHS ester and the modifier bound thereto) and a pH of 6.0 or less. The second aqueous solution may be provided in the additional container 213 separate from the multi-arm PEG having succinimidyl ester tethering groups and the at least one modifier including a catechol moiety, such as to prevent premature, unwanted, or incomplete reactions of the components therein. In some examples, the additional container 213 may include a vial, sample container, or the like.

The kit 200 may include a delivery or mixing means configured to at least deliver the hydrogel embolic to a selected location in the human body. For example, the kit 200 may include the dual lumen connector 252 having connectors 253 and 255 configured to fluidly connect to the first container 211 and the second container 221, respectively. The connectors 253 and 255 may be configured to fluidly connect (e.g., mix, react) the first group of components with the second group of components to form the hydrogel embolic. For example, the dual lumen connector 252 may be configured to fluidly connect to the first syringe and the second syringe and combine the contents thereof at a selected point, such as in or at an output conduit 257 of the dual lumen connector 252. In such examples, the dual lumen connector may be configured as a dual lumen peripherally inserted central catheter (PICC). The PICC may have at least a 1 French size such as, 1 French to 10 French, 3 French to 7 French, 4 French to 6 French, or a 5 French size. The dual lumen connector may include a T or Y connection for connecting the output of the first and second syringes, such as in a common conduit (e.g., tube or hose).

In some examples, the kit 200 includes the syringe connector 254. The syringe connector 254 is configured to connect the internal spaces of two syringes together, such as by connecting the output (e.g., hub or needle) of a first syringe directly to the output of a second syringe (e.g., in an opposing configuration). The syringe connector 254 may be configured to allow back and forth mixing of components of two syringes from one syringe into the other syringe effective to form a substantially uniform solution from the components of the separate syringes. For example, the additional container 213 (e.g., first syringe) may be directly coupled to the first container 211 (e.g. first syringe) via the syringe connector 254 to hydrate the polyamine therein and to form the basic solution via mixing. Similarly, the additional container 213 (e.g., additional syringe) may be directly coupled to the second container 221 (e.g., second syringe) via the syringe connector 254 to hydrate the PEG-NHS ester and modifier therein and to form the acidic solution. The syringe connector 254 may include longitudinally aligned syringe interfaces sized and shaped to form a fluid tight seal with a syringe output structure (e.g., syringe hub). Accordingly, the syringe connector 254 allows the contents of syringes to be fluidly connected to each other for mixing.

The kit 200 may include a dual syringe adapter 260 configured to hold and actuate two syringes simultaneously. The dual syringe adapter 260 may include first and second syringe fixtures configured to hold and secure two syringes therein, a dispenser handle configured to promote a grip by a human of the dual syringe adapter, and an actuator for dispensing the contents of the two syringes, such as by pushing the plunges of the syringes simultaneously.

The contents of the kit 200 may provide for mixing one or more components of the hydrogel embolic and delivering the hydrogel embolic to a selected anatomical feature within the human body (e.g., veins).

The kit 200 may include packaging, such as a box, tray, bag, or the like. The sub-components (e.g., PEG-NHS ester, modifier, base, and polyamine) of the kit 200 may be provided separately in the packaging. The sub-components of the kit 200 may be provided in one or more separated indentations of a tray, in sealed individual containers, in boxes, or combinations of the foregoing. The sub-components may be mixed to form the adhesive component and the at least one complexing component. In some examples, one or more sub-components may be combined in the kit 200, such as a basic solution having polyamine and base dissolved therein in the first container. The mixed sub-components may be combined (e.g., mixed and reacted) by a user to form the hydrogel embolic.

Any of the hydrogel embolics, components thereof, or kits containing the same may be used to treat one or more vessels in the human body, such as veins for treating CVI. As shown in FIG. 1, the hydrogel embolics and techniques for forming the same in a one-pot synthesis disclosed herein may provide hydrogel embolics that bond very well with wet tissue 180 without using cyanoacrylates or other hard-setting, substantially permanent adhesives. Further, the hydrogel embolics and techniques for forming the same in a one-pot synthesis disclosed herein provide a resorbable hydrogel embolic with a tunable effective life that eliminates or at least reduces pain compared to other CVI treatments, such as cyanoacrylate embolics or thermal treatments.

The embodiments disclosed herein include treatment of vessel (e.g., veinous) conditions via embolic treatment using one or more of the hydrogel embolics disclosed herein.

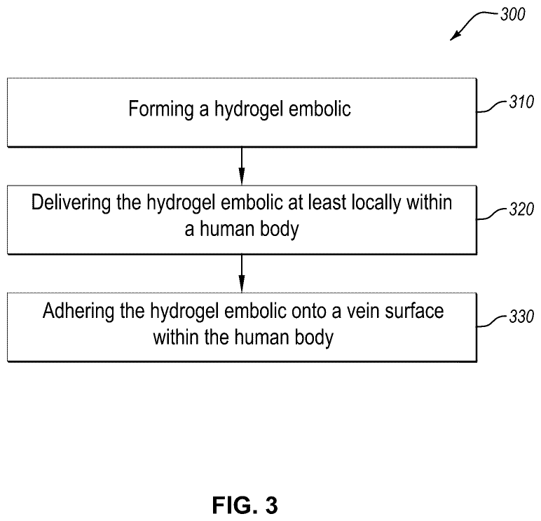

FIG. 3 is a flow chart of a method 300 of treating a veinous condition in a human body, according to an embodiment. The method 300 includes an act 310 of forming a hydrogel embolic; an act 320 of delivering the hydrogel embolic at least locally within the human body via an embolization, and an act 330 of adhering the hydrogel embolic onto a vein surface within the human body. In some examples, the method 300 may include more or fewer acts than acts 310-330. For example, the acts 320 and 330 may be combined into a single act or the act 310 may be broken into more than one act.

The act 310 of forming a hydrogel embolic may include forming any of the hydrogel embolics disclosed herein using and the components and techniques disclosed herein. The hydrogel embolic may be the hydrogel embolic. For example, the hydrogel embolic may include an adhesive component bound to the at least one complexing component.

The at least one adhesive component may include a core including a glycerol base, at least one linker (e.g., PEG) bound to the core, and at least one modifier bonded to the at least one linker, the at least one modifier including at least one catechol moiety. The at least one adhesive component may include a tethering group or reaction product thereof bonded to the at least one linker and the at least one modifier, thereby bonding the linker to the modifier. Any of the cores, linkers, tethering groups, or modifiers disclosed herein may be used to form the hydrogel embolic.

The at least one complexing component may include any of the polyamines disclosed herein. The resulting hydrogel embolic includes a hydrogel embolic having catechol moieties (from the modifier) which readily bond with wet veinous surfaces of the human body and which are resorbed in a selected amount of time based on their composition.

The act 310 of forming a hydrogel embolic may include mixing the components of the hydrogel embolics (e.g., hydrogel adhesives) disclosed herein. For example, the forming a hydrogel embolic may include mixing any of the first groups of components disclosed herein with any of the second groups of components disclosed herein. The first group of components may include the complexing component. In some examples, the first group of components may include a polyamine (e.g., albumin), a base (e.g., sodium carbonate), and water in an amount selected to form a first aqueous solution containing the polyamine and having a pH of about 8.0 or more. In such examples, at least some of the first group of components may be premixed with each other and presented as a polyamine in a first aqueous solution having a pH of about 8.0 or more (e.g., about 8.0 to about 10.0).

The second group of components may include or form the adhesive component. For example, the second group of components may include a multi-arm polyethylene glycol (PEG) having succinimidyl ester tethering groups, the at least one modifier including the at least one catechol moiety, an acid or acidifier, and water in an amount selected to provide a second aqueous solution having a pH of less than 6.0 (e.g., about 4.0 to about 6.0). In such examples, at least some of the second group of components may be premixed with each other and presented as an adhesive component including the core having a glycerol base, the at least one linker (e.g., PEG) bound to the core, and the at least one modifier bonded to the at least one linker, the at least one modifier including at least one catechol moiety in a second aqueous solution having a pH of about 8.0 or more (e.g., about 9.0 to about 10.0).

In more specific examples, forming a hydrogel embolic may include hydrating the multi-arm PEG having succinimidyl ester tethering groups and the at least one modifier including at least one catechol moiety with the second aqueous solution effective to provide a pH of about 4.0 to about 6.0 in the second solution. Such hydration may be caried out by mixing the contents of the second container 221 (FIG. 2) with the contents of the at least one additional container 213 (e.g., water). Such mixing may be carried out from a syringe-to-syringe transfer of components between the respective syringes using the syringe connector 254.

The mixing may be accomplished by actuating the dispensing device (e.g., dual syringe adapter) containing the first syringe and the second syringe, respectively containing the first group of components and the second group of components. The groups of components may mix and react to form the hydrogel embolic through a T or Y connection of a dual syringe apparatus.

The act 320 of delivering the hydrogel embolic at least locally within the human body via an embolization may include delivering the hydrogel embolic into selected region or tissue in the human body, such as a vein to treat CVI. The embolization may include injecting the hydrogel embolic into the selected tissue via a catheter, needle, Y-connector, or the like. Delivering the hydrogel embolic at least locally withing the human body may include at least partially (e.g., fully) occluding the vein with the hydrogel embolic. Delivering the hydrogel embolic at least locally within the human body may include delivering the hydrogel embolic through any of the delivery apparatuses disclosed herein, such as via any portions of the kit 200 disclosed herein. For example, delivering the hydrogel embolic at least locally within the human body may include delivering the hydrogel embolic through a dual syringe dispensing apparatus, a PICC, or both.

In some examples, forming the hydrogel embolic and delivering the hydrogel embolic are performed contemporaneously by injecting the first group of components and the second group of components into the human body via a dual syringe dispensing apparatus.

The act 330 of adhering the hydrogel embolic onto a vein surface within the human body is effective to embolize (e.g., at least partially occlude) the vein. Adhering the hydrogel embolic onto a vein surface within the human body includes bonding the catechol moiety of the modifier in the hydrogel embolic (e.g., adhesive) to tissue of the vein surface via one or more of hydrogen bonding, pi stacking (e.g., x-x stacking), mechanical stacking, hydrophobic interactions, Schiff base reactions, or Michael type additions. Bonding the hydrogel embolic onto a vein surface within the human body may include waiting for bonding reactions to take place, such as for a selected duration. The selected duration may include at least 1 second, such as 1 second to 1 day, 10 seconds to 1 minute, 1 minute to 10 minutes, 10 minutes to 30 minutes, 30 minutes to 1 hour, less than a day, or less than an hour.

In some examples, acts 310, 320, and 330 may be performed contemporaneously or simultaneously. By performing the acts 310 and 320 at least contemporaneously, the hydrogel embolic may be formed without unwanted side reaction products and may be delivered to a selected location in a human body to provide embolization to selected tissues without thermal treatment or using conventional adhesives (cyanoacrylate based adhesives) that form hard materials within the human body. The hydrogel embolics disclosed herein also provide a resorbable material that can be cleared from the body via hydrolysis and renal clearance after a selected and tunable duration.

As used herein, the term “about” or “substantially” refers to an allowable variance of the term modified by “about” by +10% or +5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A hydrogel embolic, comprising:
an adhesive component including,
a core including a glycerol base;
at least one linker bound to the core, the at least one linker including a polyethylene glycol (PEG); and
at least one modifier bonded to the at least one linker, the at least one modifier including at least one catechol moiety; and
at least one complexing component bound to the adhesive component, the at least one complexing component including a polyamine.

2. The hydrogel embolic of claim 1 wherein the polyamine includes one or more of human serum albumin, recombinant human serum albumin, bovine serum albumin, polylysine, Spermine, spermidine, Tris(2-aminoethyl)amine, polyethylenimine, chitosan, collagen, gelatin, keratin, fibroin, or elastin.

3. The hydrogel embolic of claim 1 wherein the at least one modifier includes dopamine, N-hydroxyl succinimide esters of any carboxylic acids containing a catechol or gallate moiety, such as NHS ester of caffeic acid, NHS ester of gallic acid and NHS ester of L-DOPA.

4. The hydrogel embolic of claim 1 wherein the core includes one or more of pentaerythritol, hexaglycerol, tripentaerythritol, or glycerol.

5. The hydrogel embolic of claim 1 further comprising a tethering group bound to the at least one linker, the tethering group including a succinimide.

6. The hydrogel embolic of claim 1 wherein:
the core includes one or more of pentaerythritol, hexaglycerol, tripentaerythritol, or glycerol;
the at least one modifier includes dopamine; and
the polyamine includes one or more of human serum albumin, recombinant human serum albumin, bovine serum albumin, polylysine, spermidine, spermine, Tris(2-aminoethyl)amine, polyethylenimine, chitosan, collagen, gelatin, keratin, fibroin, or elastin.

7. A kit for dispensing a hydrogel embolic into a human, the kit comprising:
a first group of components including a polyamine, sodium carbonate, and a first aqueous solution having a pH of about 8.0 or more;
a second group of components including,
a multi-arm polyethylene glycol (PEG) having succinimidyl ester tethering groups;
at least one modifier including a catechol moiety; and
a second aqueous solution.

8. The kit of claim 7 wherein the at least one modifier includes dopamine or dopamine hydrochloride.

9. The kit of claim 7 wherein the a multi-arm (PEG) having succinimidyl ester tethering groups includes an 8-arm PEG-NHS ester.

10. The kit of claim 7 wherein the polyamine includes one or more of human serum albumin, recombinant human serum albumin, bovine serum albumin, polylysine, or spermidine.

11. The kit of claim 7 wherein:
the first aqueous solution is composed to have a pH between about 8.0 and 12.0 after the first group of components are mixed; and
the second aqueous solution is composed to have a pH between about 2.0 and 6.0 after the second group of components are mixed.

12. The kit of claim 11 wherein the first aqueous solution and the second aqueous solution are composed to have a pH of at least about 9.0 after the first group of components and the second group of components are mixed with each other.

13. The kit of claim 7 further comprising a first container holding the first group of components and a second container holding the second group of components.

14. The kit of claim 13 wherein the first container includes a first syringe and the second container includes a second syringe.

15. The kit of claim 14, further comprising a dual lumen connector configured to fluidly connect to the first syringe and the second syringe.

16. The kit of claim 15 wherein the dual lumen connector includes a dual lumen peripherally inserted central catheter.

17. The kit of claim 14 further comprising a dual syringe adapter configured to hold and actuate two syringes simultaneously.

18. The kit of claim 17 further comprising a syringe connector configured to connect internal spaces of two syringes together.

19. The kit of claim 7 wherein the second aqueous solution is provided in a separate container than the multi-arm PEG having succinimidyl ester tethering groups and the at least one modifier including a catechol moiety.

20. A method of treating a veinous condition in a human body, the method comprising:
forming a hydrogel embolic, the hydrogel embolic including,
an adhesive component including,
a core including a glycerol base;
at least one linker bound to the core, the at least one linker including a polyethylene glycol (PEG); and
at least one modifier bonded to the at least one linker, the at least one modifier including at least one catechol moiety;
at least one complexing component bound to the adhesive component, the at least one complexing component including a polyamine;
delivering the hydrogel embolic at least locally within the human body via an embolization; and
adhering the hydrogel embolic onto a vein surface within the human body.

21. The method of claim 20 wherein:
forming a hydrogel embolic includes mixing a first group of components with a second group of components;
the first group of components includes a polyamine, sodium carbonate, and a first aqueous solution having a pH of about 8.0 or more; and
the second group of components includes,
a multi-arm polyethylene glycol (PEG) having succinimidyl ester tethering groups;
the at least one modifier including the at least one catechol moiety; and
a second aqueous solution having a pH of about 2.0 to about 6.0.

22. The method of claim 21 wherein forming a hydrogel embolic includes hydrating the multi-arm PEG having succinimidyl ester tethering groups and the at least one modifier including at least one catechol moiety with the second aqueous solution effective to provide a pH of about 2.0 to about 6.0 in the second solution.

23. The method of claim 21 wherein forming the hydrogel embolic and delivering the hydrogel embolic are performed contemporaneously by injecting the first group of components and the second group of components into the human body via a dual syringe dispensing apparatus.

24. The method of claim 20 wherein adhering the hydrogel embolic onto a vein surface within the human body includes bonding the at least one catechol moiety to a vein surface of the human body via one or more of hydrogen bonding, hydrophobic interactions, Schiff base reactions, Michael-type reactions, Pi-stacking, or mechanical stacking.
